(19) United States
(12) Patent Application Publication
(10) Pub. No.: US 2026/0294457 A1
(43) Pub. Date: Oct. 1, 2026

(54) INSTRUMENT CONNECTION SYSTEMS AND METHODS

(71) Applicant: ECA Medical Instruments, Thousand Oaks, CA (US)

(72) Inventors: David Skinlo, Thousand Oaks, CA (US); Dan Avni-Baifus, Thousand Oaks, CA (US); Zachary Hixson, Thousand Oaks, CA (US)

(21) Appl. No.: 19/631,867

(22) Filed: Mar. 27, 2026

Related U.S. Application Data
(60) Provisional application No. 63/779,067, filed on Mar. 27, 2025.

(51) Int. Cl. A61B 17/28; A61B 10/02; A61B 17/00; A61B 17/3201; A61B 90/00

(52) U.S. Cl. CPC A61B 17/2841; A61B 10/025; A61B 17/282; A61B 17/2833; A61B 17/3201; A61B 90/06; A61B 2017/00407; A61B 2017/00477; A61B 2017/2837; A61B 2090/061

(57) ABSTRACT

A modular forceps system is disclosed for coupling interchangeable tool tips to reusable or disposable forceps handles. The system may include handles having a receiving portion and tool tips having corresponding mating components configured for connection within the receiving portion. Cooperative interface features between the handles and tool tips constrain relative movement in multiple degrees of freedom, including rotational, axial, and lateral directions. In certain embodiments, a locking mechanism, such as a spring-biased element engaging a corresponding recess, retains the tool tips within the handles. The mating components may include non-circular geometries to inhibit rotation and provide alignment. The handle may be formed from polymeric materials, while the tool tips may be formed from metal or other durable materials. The system enables a standardized interface for a variety of surgical or medical tool tips, reducing manufacturing cost, improving versatility, and facilitating disposable or semi-disposable instrument configurations.

### CROSS-REFERENCE TO RELATED APPLICATION

This application claims the priority benefit of U.S. Provisional Pat. App. No. 63/779,067 to Skinlo, et al., filed Mar. 27, 2025 and entitled “Locking Connection for Forceps,” which is fully incorporated by reference herein in its entirety.

### BACKGROUND OF THE DISCLOSURE

### Field of the Disclosure

The present disclosure generally relates to modular connection methods for releasably or permanently coupling one or more components to another. More specifically, this disclosure relates to forceps, such as those for use in medical procedures.

### Description of the Related Art

In many industries, it is desirable to selectively couple interchangeable components using a secure connection that resists movement in multiple degrees of freedom. Such systems may be useful in medical, dental, laboratory, industrial, consumer, cosmetic, cleaning, food-service, precision assembly, or maintenance applications. Conventional assemblies often require dedicated unitary devices or fail to provide a standardized interface that permits secure interchangeability or modularity.

For example, medical and surgical procedures frequently require the use of specialized instruments designed for cutting, grasping, retracting, or otherwise manipulating tissue. In many instances, the tools used in such applications are attached to forceps to provide for secure attachment to bone, tissue, etc. Traditionally, such instruments are manufactured as unitary devices in which the functional metal working end is permanently affixed to a dedicated handle. These instruments are commonly fabricated from stainless steel or other durable metals to withstand repeated sterilization and use. While effective, this construction results in relatively high manufacturing costs due to the material requirements, machining processes, and assembly involved.

In recent years, there has been increased interest in reducing costs and improving efficiency in medical settings, particularly with respect to single-use or limited-use instruments. However, many existing instruments remain expensive to produce and procure, especially when each variation of a tool requires a fully assembled, standalone device. This can lead to increased inventory burdens, as healthcare providers must stock multiple complete instruments that differ only in the configuration of their functional, tool end.

Contemporary medical tool systems have not achieved widespread standardization. The lack of a common interface for coupling instrument components limits flexibility and prevents cost savings that could otherwise be realized through interchangeable parts.

Accordingly, there exists a need for an improved system that allows a variety of metal instrument working ends to be selectively and securely attached to a standardized handle. In particular, a system utilizing a standardized plastic forceps handle could reduce material costs, simplify manufacturing, and enable disposable or semi-disposable use, while still maintaining the strength and precision required of the metal working components. A standardized attachment mechanism would further promote interoperability, reduce inventory complexity, and provide a more economical solution for healthcare providers.

### SUMMARY OF THE DISCLOSURE

Disclosed herein are systems, methods, and devices for connecting a first component to a second component, such as connecting a working tool to a handle.

One embodiment of a modular instrument system according to the present disclosure includes locking scissor handles each including a receiving portion and a first mating component within the receiving portion; and tool tips each including a working end and a second mating component opposite the working end. The first mating component and the second mating component can be configured to constrain relative movement between the locking scissor handles and the tool tips in at least one degree of freedom.

One embodiment of a tool tip for use with a modular instrument according to the present disclosure includes a working end and a mating component configured to be removably coupled to a handle. The mating component can include at least one interface feature configured to engage a complementary feature of the handle to constrain relative movement between the tool tip and the handle.

One embodiment of a modular connection system according to the present disclosure includes a first component, which may include a receiving portion and a first mating component within the receiving portion, and a second component, which can include a second mating component configured to engage the first mating component to secure the second component within the receiving portion of the first component.

This has outlined, rather broadly, the features and technical advantages of the present disclosure so that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further features and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIGS. 1A-1D show various views of an embodiment of forceps incorporating features of the present disclosure;

FIGS. 2A-2B show various views of another embodiment of forceps incorporating features of the present disclosure;

FIGS. 3A-3B show various views of another embodiment of forceps incorporating features of the present disclosure;

FIG. 4 shows a top, perspective view of another embodiment of forceps incorporating features of the present disclosure;

FIG. 5 shows a top, perspective view of another embodiment of forceps incorporating features of the present disclosure;

FIGS. 6A-6B show various views of another embodiment of forceps incorporating features of the present disclosure;

FIG. 7 shows a top, perspective view of another embodiment of forceps incorporating features of the present disclosure;

FIG. 8 shows a top, perspective view of another embodiment of forceps incorporating features of the present disclosure;

FIGS. 9A-9B show various views of another embodiment of forceps incorporating features of the present disclosure;

FIGS. 10A-10B show various views of another embodiment of forceps incorporating features of the present disclosure;

FIGS. 11A-11B show various views of another embodiment of forceps incorporating features of the present disclosure;

FIG. 12 shows a top, left-side perspective view of yet another embodiment of forceps incorporating features of the present disclosure.

### DESCRIPTION OF THE DISCLOSURE

In the following detailed description, numerous specific details are set forth in order to provide a more thorough understanding of embodiments incorporating features of the present disclosure. However, it will be apparent to one skilled in the art that devices and methods according to the present disclosure can be practiced without necessarily being limited to these specifically recited details.

Throughout this disclosure, embodiments and examples illustrated should be considered as exemplars, rather than as limitations on the present disclosure. As used herein, the term “invention,” “device,” “method,” “present invention,” “present device,” or “present method” refers to any one of the embodiments of the disclosure described herein, and any equivalents. Furthermore, reference to various feature(s) of the “invention,” “device,” “method,” “present invention,” or “present device,” or “present method” throughout this document does not mean that all claimed embodiments or methods must include the referenced feature(s).

Furthermore, any element in a claim that does not explicitly state “means for” performing a specified function, or “step for” performing a specific function, is not to be interpreted as a “means” or “step” clause as specified in 35 U.S.C. § 112, for example, in 35 U.S.C. § 112(f) or pre-AIA 35 U.S.C. § 112, sixth paragraph. In particular, the use of “step of” or “act of” in the claims herein is not intended to invoke the provisions of 35 U.S.C. § 112.

It is also understood that when an element or feature is referred to as being “on” or “adjacent” to another element or feature, it can be directly on or adjacent the other element or feature or intervening elements or features may also be present. It is also understood that when an element is referred to as being “attached,” “connected” or “coupled” to another element, it can be directly attached, connected or coupled to the other element or intervening elements may be present. In contrast, when an element is referred to as being “directly attached,” “directly connected” or “directly coupled” to another element, there are no intervening elements present.

Furthermore, relative terms such as “left,” “right,” “front,” “back,” “top,” “bottom” “forward,” “reverse,” “clockwise,” “counter-clockwise,” “outer,” “inner,” “above,” “upper,” “lower,” “below,” “horizontal,” “vertical,” and similar terms, have been used for convenience purposes only and are not intended to imply any particular fixed direction. Instead, they are used to describe a relationship of one element to another. Terms such as “higher,” “lower,” “wider,” “narrower,” and similar terms, may be used herein to describe angular relationships. It is understood that these terms are intended to encompass different orientations of the elements or system in addition to the orientation depicted in the figures.

Although ordinal terms, e.g., first, second, third, etc., may be used herein to describe various elements or components, these elements or components should not be limited by these terms. These terms are only used to distinguish one element or component from another element or component. Thus, a first element or component discussed below could be termed a second element or component without departing from the teachings of the present disclosure.

The terminology used herein is for describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Embodiments as described in the present disclosure can be described herein with reference to view illustrations that are schematic in nature. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the disclosure. Further, it is understood that, while embodiments of the present disclosure comprise various shapes, these shapes are not exhaustive, and other shapes are possible.

It is understood that when a first element is referred to as being “between” or “interposed between” two or more other elements, the first element can be directly between the two or more other elements or intervening elements may also be present between the two or more other elements. For example, if a first element is “between” or “interposed between” a second and third element, the first element can be directly between the second and third elements with no intervening elements, or the first element can be adjacent to one or more additional elements with the first element and these additional elements all between the second and third elements.

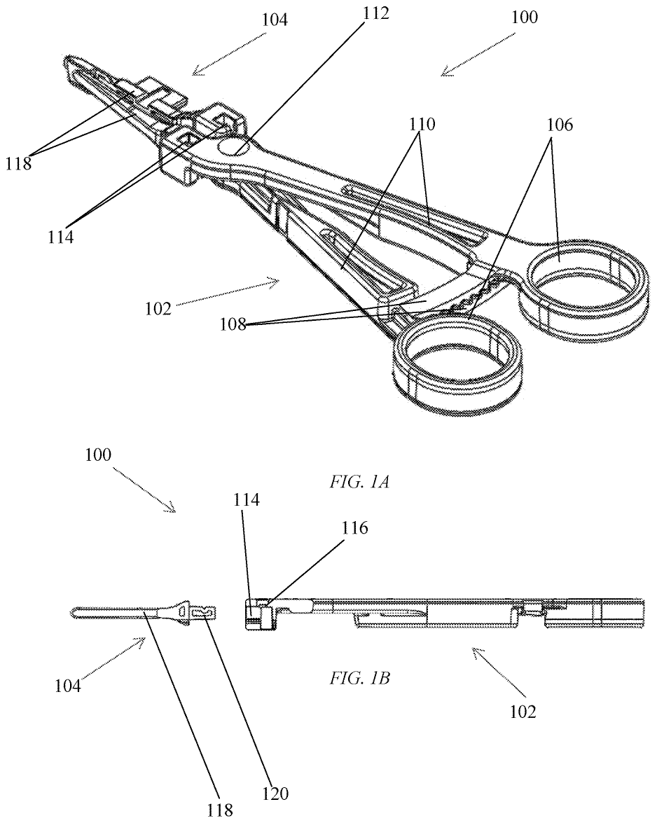

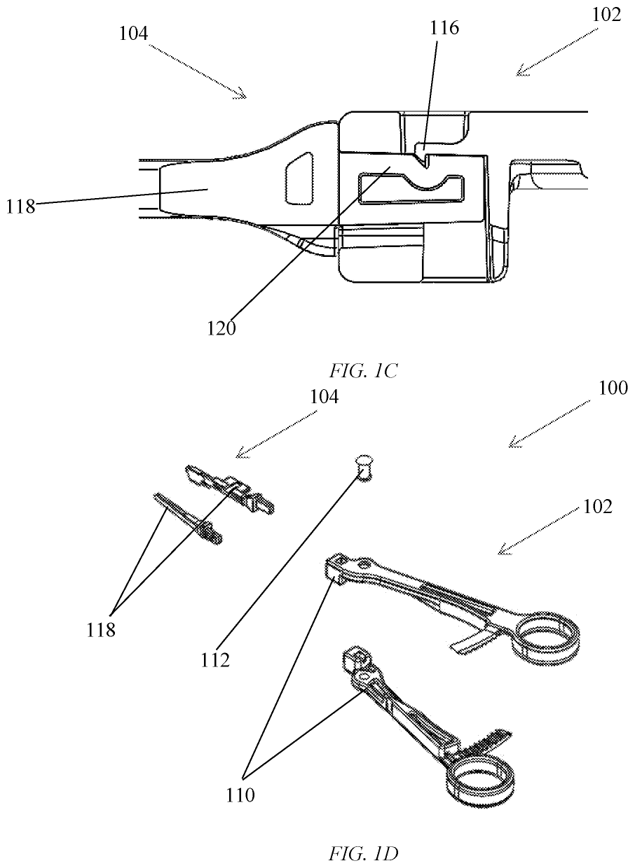

FIGS. 1A-1D show one embodiment of a forceps system 100 according to the present disclosure. The forceps system 100 can include locking scissor handles 102 and tool tips 104. In some embodiments, the locking scissor handles 102 may be made of a disposable material, such as for example, polypropylene, polyethylene, polystyrene, acrylonitrile butadiene styrene, polycarbonate, polyvinyl chloride, polyetherimide, nylon, polyether ether ketone, thermoplastic elastomers, glass-filled or carbon fiber reinforced polymers, biodegradable materials, and other suitable materials known in the art, or combinations of the foregoing. The tool tips 104 may be made of stainless steel, titanium, cobalt, nickel, polymers, and other materials known in the art, or combinations of the foregoing.

As shown in FIG. 1A, the locking scissor handles 102 may include finger rings 106, ratcheting components 108, shafts 110, a rivet 112, and slots 114, and (as shown in FIGS. 1B-1C) mating components 116. The tool tips 104 can include a tool 118 and a mating component 120. In some embodiments, a portion of the tool tip, such as for example the mating component 120, may be inserted into the slot 114 of the locking scissor handles 102. The mating component 120 of the tool tip 104 can then mate with a corresponding mating feature 116 of the locking scissor handle 102. As shown in FIGS. 1A-1D, the mating components 120, 116 may be, for example, a notch and spring-tensioned finger designed to deflect while the tool tip 104 is being inserted and to, upon reaching the notch, snap into the notch to retain the tool tip 104. Doing so creates a stable and connected assembly that ensures a secure connection between the components resulting in an assembly that behaves homogeneously.

As used herein “slot” may refer to any receiving portion, receptacle, opening, recess, slot, channel, bore, socket, cavity, passage, or other structure configured to receive at least a portion of a mating component or body portion of a corresponding component. Such a slot or receiving portion may be open or partially enclosed, may extend fully or partially through a body, and may have any suitable depth, profile, or cross-sectional shape. Such a slot or receiving portion may also guide, align, support, and/or position the corresponding component and may cooperate with one or more interface features to constrain relative movement between components.

The forceps system 100 can prevent movement of the tool tips 104 relative to the locking scissor handles 102 in various degrees of freedom via three design features. First, restricting rotation of the components relative to each other is achieved by the rectangular shape of the mating components and corresponding rectangular shape of the slot 114. Restriction of toggle or bending relative to each other is achieved by a tight fit between two respective rectangular mating components 120,116. Linear motion relative to each other is achieved by a locking clip 116 on the locking scissor handles 102 that mates with a notch in the tool tips 104.

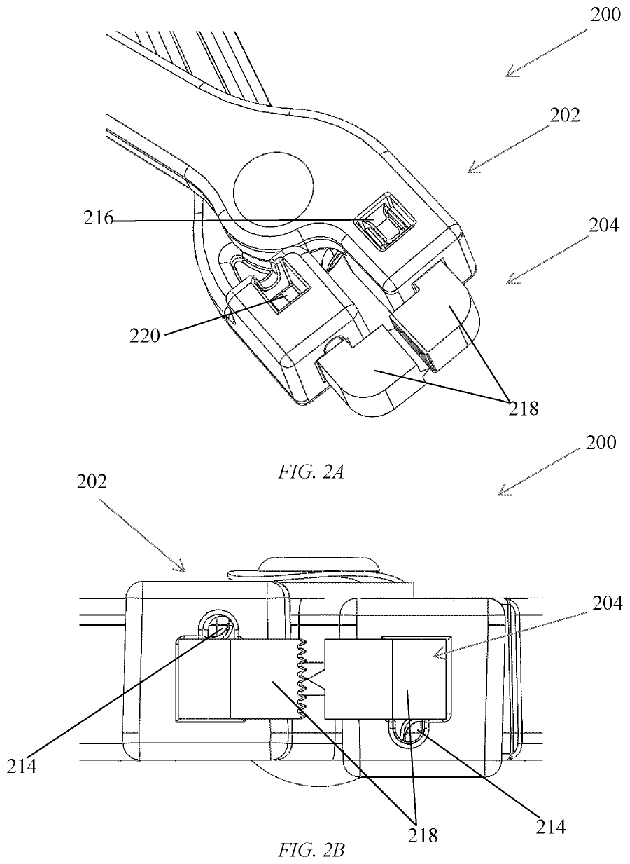

The tools 118 shown in FIGS. 1A-1D are a shovel and shaft configuration as an example, but it is understood that many different tool tips are possible, such as those discussed below with respect to FIGS. 2A-12.

FIGS. 2A-2B show another forceps system 200 according to an embodiment of the present disclosure. Like the forceps system 100, the system 200 includes locking scissor handles 202 and tool tips 204. The tool tips 204 are configured with bone biopsy tips 218, which include a sharp point for cutting and breaking bones, then removing bone fragments. Visible in FIG. 2A are a mating feature 216 on the locking scissor handles 202 and the bottom of a mating feature 220 of the tool tip 204. Although not pictured, the tool tips 204 can contain mating components 220 similar to or identical to the mating components 120, which can mate with corresponding mating components 216 on the locking scissor handles 202.

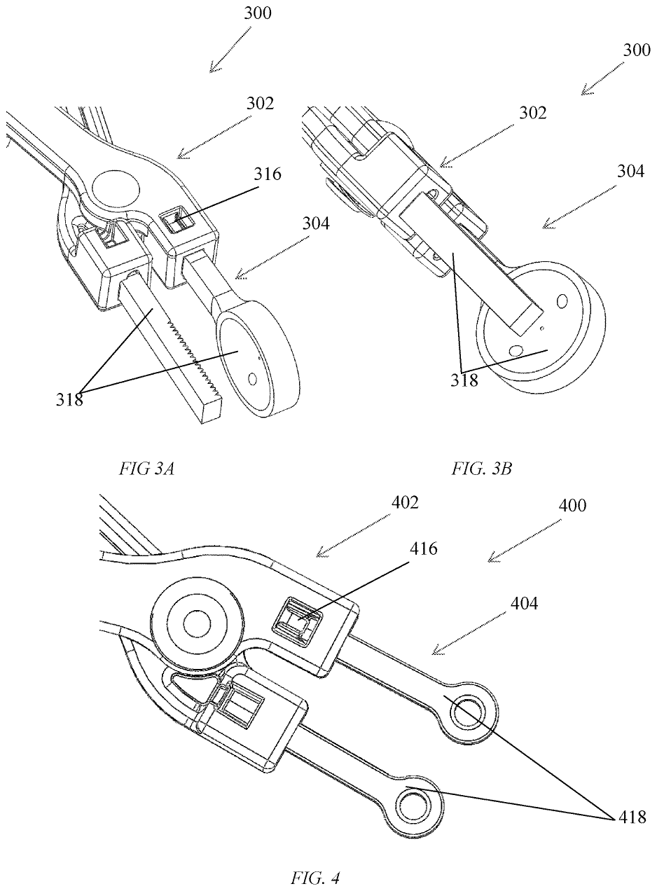

FIGS. 3A-3B show another forceps system 300 according to an embodiment of the present disclosure. Like the forceps system 100,200, the system 300 includes locking scissor handles 302 and tool tips 304. The tool tips 304 are configured with bone clamping and drilling tips 318, which include a toothed shaft and dished tip, which may be used for clamping and placing a k-wire or drilling a hole in preparation for, e.g., patella surgery. Although not pictured, the tool tips 304 can contain mating components 320 similar to or identical to the mating components 120,220, which can mate with corresponding mating components 316 (not pictured) on the locking scissor handles 302.

FIG. 4 shows another forceps system 400 according to an embodiment of the present disclosure. Like the forceps system 100,200,300 the system 400 includes locking scissor handles 402 and tool tips 404. The tool tips 404 are configured with drill guide and distraction/compression tips 418. The tips 418 contain holes that can translate to a set position for use as a drill guide. A k-wire can be inserted in either hole to distract or compress bone, depending on the application. Although not pictured, the tool tips 404 can contain mating components 420 similar to or identical to the mating components 120,220,320 which can mate with corresponding mating components 416 on the locking scissor handles 402.

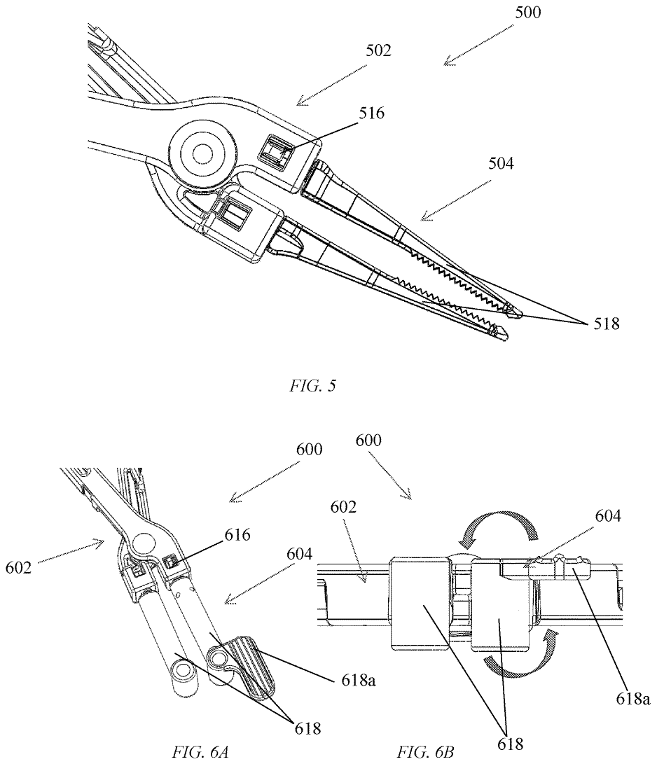

FIG. 5 shows another forceps system 500 according to an embodiment of the present disclosure. Like the forceps system 100,200,300,400 the system 500 includes locking scissor handles 502 and tool tips 504. The tool tips 504 are configured with jaw tips 518. The tips 518 contain serrated portions that can be used for general grasping and manipulation of tissue, bone, other instruments, etc. Although not pictured, the tool tips 504 can contain mating components 520 similar to or identical to the mating components 120,220,320,420 which can mate with corresponding mating components 516 on the locking scissor handles 502.

FIGS. 6A-6B show another forceps system 600 according to an embodiment of the present disclosure. Like the forceps system 100,200,300,400,500 the system 600 includes locking scissor handles 602 and tool tips 604. The tool tips 604 are configured with Lapidus tips 618. The tips 618 allow for distraction and rotation of attached joints, as a Lapidus instrument. Although not pictured, the tool tips 604 can contain mating components 620 similar to or identical to the mating components 120,220,320,420,520 which can mate with corresponding mating components 616 on the locking scissor handles 602.

FIG. 7 shows another forceps system 700 according to an embodiment of the present disclosure. Like the forceps system 100,200,300,400,500,600 the system 700 includes locking scissor handles 702 and tool tips 704. The tool tips 704 are configured with pin removal tips 718. The tips 718 allow for removal of pins, wires, and other features by clamping such features within a slot and tang. Other embodiments include pin removal tips 718 rotated 90 degrees from those shown in FIG. 7. Many variations are possible, as one of skill in the art would appreciate. Although not pictured, the tool tips 704 can contain mating components 720 similar to or identical to the mating components 120,220,320,420,520,620 which can mate with corresponding mating components 716 on the locking scissor handles 702.

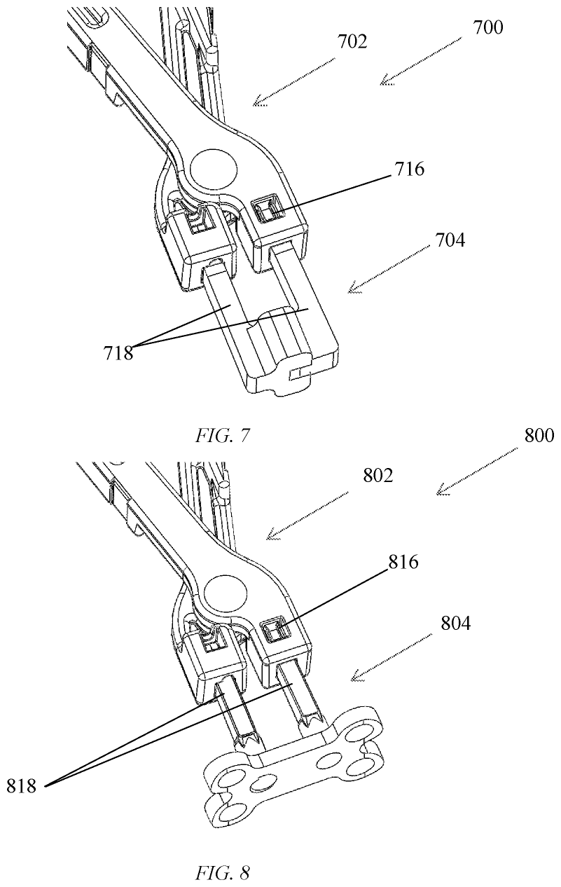

FIG. 8 shows another forceps system 800 according to an embodiment of the present disclosure. Like the forceps system 100,200,300,400,500,600,700 the system 800 includes locking scissor handles 802 and tool tips 804. The tool tips 804 are configured with fracture plate and cervical plate positioning tips 818. The tips 818 include connecting fingers that engage with a plate, allowing for placement thereof prior to final fixation and repair. In some embodiments, the connecting fingers can be specific to a certain plate. It is understood that many geometries are possible for the connecting finger, e.g., round posts (as shown in FIG. 8), square, triangle, other regular or irregular polygons, and other shapes known in the art. The plate shown in FIG. 8 is exemplary only. Many are possible. Although not pictured, the tool tips 804 can contain mating components 820 similar to or identical to the mating components 120,220,320,420,520,620,720 which can mate with corresponding mating components 816 on the locking scissor handles 802.

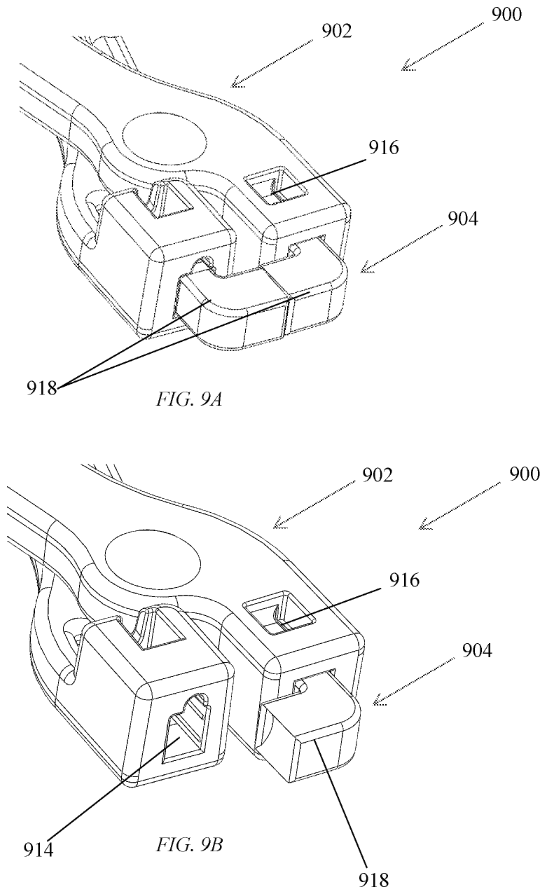

FIGS. 9A-9B show another forceps system 900 according to an embodiment of the present disclosure. Like the forceps system 100,200,300,400,500,600,700,800 the system 900 includes locking scissor handles 902 and tool tips 904. The tool tips 904 are configured with tissue biopsy tips 918. The tips 918 can function similar to a rongeur for tissue removal and sampling. In some embodiments, the tips 918 can include a recess, or “cup” within the surfaces of the tips 918 that contact the other tip to retain removed tissue. It is understood that many different recess shapes are possible. Although not pictured, the tool tips 904 can contain mating components 920 similar to or identical to the mating components 120,220,320,420,520,620,720,820 which can mate with corresponding mating components 916 on the locking scissor handles 902.

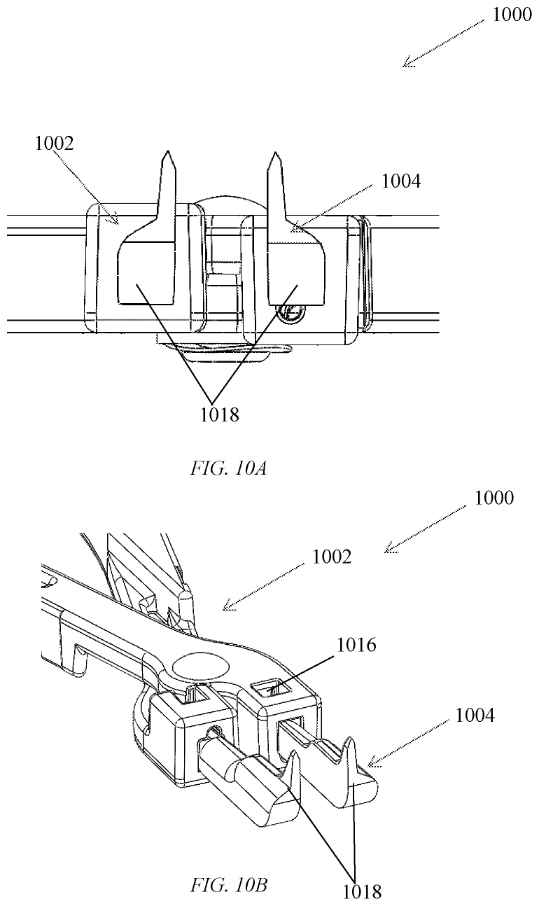

FIGS. 10A-10B show another forceps system 1000 according to an embodiment of the present disclosure. Like the forceps system 100,200,300,400,500,600,700,800,900 the system 1000 includes locking scissor handles 1002 and tool tips 1004. The tool tips 1004 are configured with measurement tips 1018. Like calipers, the system 1000 can measure length by compressing or expanding. In specific embodiments, the locking scissor handles 1002 include measurements along ratcheting components 1008. Although not pictured, the tool tips 1004 can contain mating components 1020 similar to or identical to the mating components 120,220,320,420,520,620,720,820,920 which can mate with corresponding mating components 1016 on the locking scissor handles 1002.

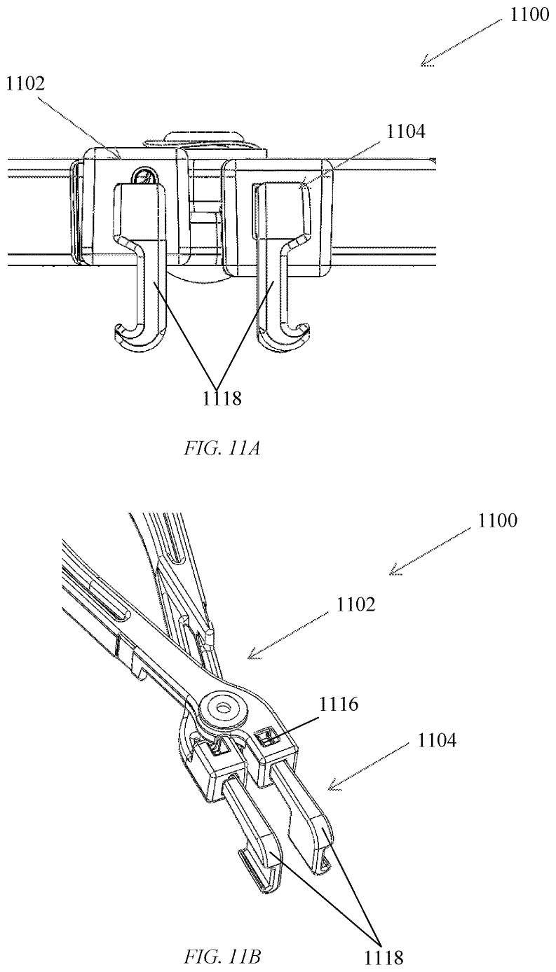

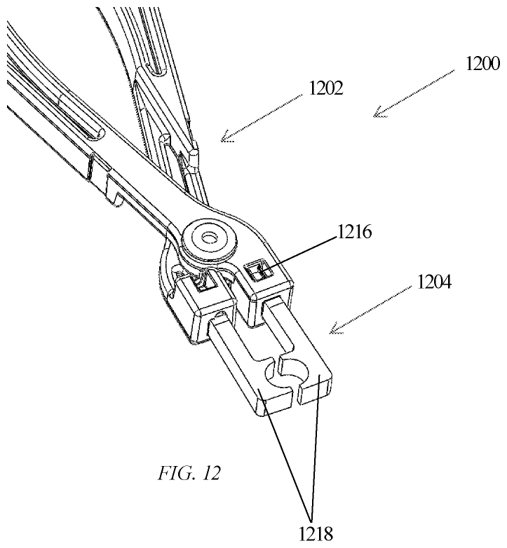

FIGS. 11A-11B show another forceps system 1100 according to an embodiment of the present disclosure. Like the forceps system 100,200,300,400,500,600,700,800,900,1000 the system 1100 includes locking scissor handles 1102 and tool tips 1104. The tool tips 1104 are configured with tissue retraction tips 1118. The tips 1118 can loop under skin or other soft tissue to distract an incision and increase visibility. It is understood that many geometries are possible, such as for example a “rake finger.” Additionally, thickness, length, etc. may be varied for various applications. Although not pictured, the tool tips 1104 can contain mating components 1120 similar to or identical to the mating components 120,220,320,420,520,620,720,820,920,1020 which can mate with corresponding mating components 1116 on the locking scissor handles 1102.

FIG. 12 shows another forceps system 1200 according to an embodiment of the present disclosure. Like the forceps system 100,200,300,400,500,600,700,800,900,1000,1100 the system 1200 includes locking scissor handles 1202 and tool tips 1204. The tool tips 1204 are configured with rod or static instrument positioning tips 1218. The tips 1218 can hold a rod or instrument at a fixed position. Although not pictured, the tool tips 1204 can contain mating components 1220 similar to or identical to the mating components 120,220,320,420,520,620,720,820,920,1020,1120 which can mate with corresponding mating components 1216 on the locking scissor handles 1202.

As one of skill would appreciate after having reviewed the foregoing figures, many tool tips 104,204,304,404,504,604,704,804,904,1004,1104,1204 may be manufactured with different tool tips 118,218,318,418,518,618,718,818,918,1018,1118,1218, but substantially identical mating components. Many locking scissor handles can be manufactured in a standardized format with mating components that can mate with the tool tip mating components. Such systems simplify manufacturing and reduce costs associated with medical instruments. Additionally, in some embodiments, the locking scissor handles can be disposable or semi-disposable to reduce or eliminate sterilization burdens.

In some embodiments, a common interface standard may be shared across a family of locking scissor handles and tool tips such that multiple different tool tips are selectively compatible with one or more standardized locking scissor handles. The disclosed systems may therefore be provided as kits, sets, or platforms of interoperable components.

It is generally understood that one or both mating components may comprise one or more features configured to engage one another and to constrain relative movement between corresponding components, including at least rotational movement. While finger shaped geometries are used herein as examples, it is understood that other features and noncircular geometries are possible, such as for example, polygonal, keyed, splined, or otherwise irregular cross-sectional configurations, such geometries being configured to inhibit rotational movement between the instrument and the handle, or more generally between the second component and the first component. In some embodiments, the mating components may comprise complementary features configured to engage one another, including protrusions, recesses, keyed surfaces, splines, flats, grooves, or combinations thereof. The mating components may be formed integrally with the locking scissor handles 102 and tool tips 104, respectively, or more generally with the second component and the first component, or may comprise separate components coupled thereto.

The mating components may constrain one or more modes of relative movement, including rotation, axial translation, lateral translation, pivoting, rocking, or combinations thereof. Such constraint may be achieved by complementary geometry, surface engagement, friction, interference, latching, detent engagement, threaded engagement, bayonet engagement, magnetic attraction, or any combination thereof.

In various embodiments, either of the first and second components may define the receiving portion, and either of the first and second components may define the mating portion. The particular allocation of such features between components is not limiting.

In some embodiments, the interface between the tool tip and the locking scissor handle is configured to restrict movement in multiple degrees of freedom, including rotational, lateral, and axial movement. Such restriction may be achieved through any combination of geometric interlocking, surface engagement, frictional forces, or mechanical locking features, and need not be limited to the specific structures illustrated herein.

Similarly, while a finger and notch configuration is disclosed as an example, other mechanisms are possible to resist axial separation of the tool tip from the locking scissor handle, including but not limited to clips, detents, interference fits, snap-fit features, latches, spring-biased elements, threaded engagements, bayonet connections, frictional interfaces, or combinations thereof. The locking feature may be integral with the locking scissor handle, the tool tip, or provided by cooperative engagement between features of both.

The connection systems and methods disclosed herein may be used with medical instruments, dental instruments, grooming tools, cosmetic applicators, cleaning implements, laboratory tools, inspection devices, hand tools, precision tools, consumer products, kitchen implements, or other devices in which a functional member is coupled to a support member, handle, holder, or receiver.

The working portion of the tool tip or second component may comprise a cutting feature, gripping feature, scraping feature, probing feature, applicator feature, cleaning feature, polishing feature, mixing feature, dispensing feature, sensing feature, or other functional structure.

It is understood that embodiments herein may be used in disposable, reusable, or semi-disposable systems. As such, the disclosed connection may be configured for removable coupling, releasable coupling, replaceable coupling, semi-permanent coupling, or permanent coupling.

It is understood that embodiments presented herein are meant to be exemplary. Although the present disclosure has been described in detail with reference to certain preferred configurations thereof both in the specification and in the claims, other versions are possible. Embodiments of the present disclosure can comprise any combination of compatible devices/features described herein and/or shown in the figures, and these embodiments should not be limited to those expressly illustrated and discussed. For instance and not by way of limitation, the appended claims could be modified to be multiple dependent claims so as to combine any combinable combination of elements within a claim set, or from differing claim sets. Claims depending on one independent claim could be modified so as to depend from a different independent claim. Therefore, the spirit and scope of the disclosure should not be limited to the versions described above.

While the foregoing written description of the disclosure enables one of ordinary skill to make and use what is considered presently to be the best mode thereof, those of ordinary skill will understand and appreciate the existence of variations, combinations, and equivalents of the specific embodiments, methods, systems, and examples herein. The disclosure should therefore not be limited by the above-described embodiments, methods, systems, and examples. Furthermore, certain terminology has been used for the purposes of descriptive clarity, and not to limit the present disclosure. It is therefore intended that the following appended claims include all such alterations, modifications and permutations as fall within the true spirit and scope of the present disclosure. No portion of the disclosure is intended, expressly or implicitly, to be dedicated to the public domain if not set forth in the claims.

We claim:

1. A modular instrument system comprising:
a pair of locking scissor handles, each of said handles comprising a receiving portion and a first mating component within said receiving portion; and
a pair of tool tips, each of said tool tips comprising a working end and a second mating component opposite said working end,
wherein said first mating component and said second mating component are configured to constrain relative movement between said locking scissor handles and said tool tips in at least one degree of freedom.

2. The system of claim 1, wherein said first mating component and said second mating component constrain rotational movement of said tool tips relative to said locking scissor handles.

3. The system of claim 1, wherein said locking scissor handles comprise locking components configured to prevent movement of each locking scissor handle relative to the other locking scissor handle in a direction.

4. The system of claim 1, wherein at least one of said first mating component and said second mating component comprise a non-circular geometry.

5. The system of claim 4, wherein said non-circular geometry is rectangular.

6. The system of claim 1, wherein at least one of said first mating component and said second mating component comprises a locking mechanism configured to retain said tool tips in said locking scissor handles.

7. The system of claim 6, wherein said locking component comprises a spring-biased element.

8. The system of claim 7, wherein said spring-biased element is configured to engage a recess formed in at least one of said locking scissor handles and said tool tip.

9. The system of claim 1, wherein said locking scissor handles comprise a polymer.

10. The system of claim 1, wherein said tool tips comprise a metal.

11. A tool tip for use with a modular instrument comprising:
a working end; and
a mating component configured to be removably coupled to a handle,
wherein said mating component comprises at least one interface feature configured to engage a complementary feature of said handle to constrain relative movement between said tool tip and said handle.

12. The tool tip of claim 11, wherein said mating component comprises a protrusion configured to engage a recess of said handle.

13. The tool tip of claim 11, wherein said working end comprises a cutting feature.

14. The tool tip of claim 11, wherein said working end comprises a grasping feature.

15. The tool tip of claim 11, wherein said working end comprises a measurement feature.

16. The tool tip of claim 11, wherein said working end comprises a biopsy feature.

17. A modular connection system comprising:
a first component comprising a receiving portion and a first mating component within said receiving portion;
a second component comprising a second mating component configured to engage said first mating component to secure said second component within said receiving portion of said first component.

18. The system of claim 17, wherein said first mating component and said second mating component comprise complementary features configured to restrict the relative movement of said second component relative to said second component.

19. The system of claim 18, wherein said relative movement comprises at least one of axial, rotational, and lateral movement.

20. The system of claim 17, wherein one of said first component and said second component comprise a polymer material, and
wherein the other of said first component and said second component comprise a metal.
